(19) United States
(12) Patent Application Publication
(10) Pub. No.: US 2023/0364422 A1
(43) Pub. Date: Nov. 16, 2023

(54) Deep Brain Stimulation System with Wireless Power

(71) Applicant: Sensoria Therapeutics, Inc., Irvine, CA (US)

(72) Inventor: Ross Tsukashima, Irvine, CA (US)

(73) Assignee: Sensoria Therapeutics, Inc., Irvine, CA (US)

(21) Appl. No.: 17/741,205

(22) Filed: May 10, 2022

(51) Int. Cl. A61N 1/36; A61N 1/05; A61N 1/04; A61N 5/06

(52) U.S. Cl. CPC A61N 1/36067; A61N 1/0504; A61N 1/0492; A61N 5/0622; A61N 5/0601

(57) ABSTRACT

A system for applying stimulation to the brain and obtaining electrical signals from the brain. The system includes a control system, a power source, and power coupling that can be placed on the scalp of a patient, a first, surface patch electrode which can be placed superficially or subcutaneously on the scalp of a patient, a second, subcutaneous electrode or NFC tag configured for placement under the scalp of the patient, an implant with an electrode array or LED configured for implantation in the brain, and an electrical wire connecting the second electrode and the implant, operable as both a conductor for delivery of power to the implant and delivery of sensor signals from the implant.

### FIELD OF THE INVENTIONS

The inventions described below relate to devices and methods that provide treatment of movement disorders.

### BACKGROUND OF THE INVENTIONS

Deep brain stimulation (DBS) technology has shown promise for treatment of movement and effective disorders such as Parkinson's disease, epilepsy, essential tremor and dystonia. Typical protocols use multiple sensor probes and stimulation probes attached to wiring that is connected to an implantable pulse generator. The sensor probes are implanted in the brain, and the pulse generator is implanted under the skin of the chest or abdomen, and the two are connected by an insulated wire that runs below the skin, from the head, down the side of the neck, behind the ear, to the pulse generator. Thus, the current method entails surgical implantation of wires and components under the skin of the patient, in addition to the surgical implantation of probes and leads in the brain of the patient.

### SUMMARY

The devices and methods described below provide for improved treatment of movement disorders using sensor probes and stimulation probes operated by an external control system. The system includes a control system, a power source, and power coupling that can be placed on the scalp of a patient, an intracranial electrode, surface patch electrode which can be placed superficially on, or subcutaneously under, the scalp of a patient, a second, subcutaneous electrode configured for placement under the scalp of the patient, an implantable probe with an electrode array or other stimulation mechanism configured for implantation in the brain, and an electrical wire connecting the second electrode and the implant, operable as a conductor for delivery of power to the implant and delivery of sensor signals from the implant. The system includes a control system operable to deliver power to the implant through a circuit comprising the power coupling, the intracranial electrode, brain tissue of the patient to the probe (with no wired connection between the intracranial electrode and the probe), a wired connection to the subcutaneous electrode and through the subcutaneous scalp tissue to the patch electrode and hence to the power coupling. The system may be optionally operable to receive sensor signals from the implant, through the same circuit pathway, and provide bi-directional communications between the control system and the implantable probe through the inductive coupling. The system is operable to deliver therapeutic and/or diagnostic stimulation to the brain, and also to obtain diagnostic information from the brain.

### BRIEF DESCRIPTION OF THE DRAWINGS

FIG. 1 illustrates a system for diagnosis and treatment of movement disorders using sensor probes and stimulation probes operated by an external control system.

FIG. 2 illustrates the probe and subcutaneous electrode.

### DETAILED DESCRIPTION OF THE INVENTIONS

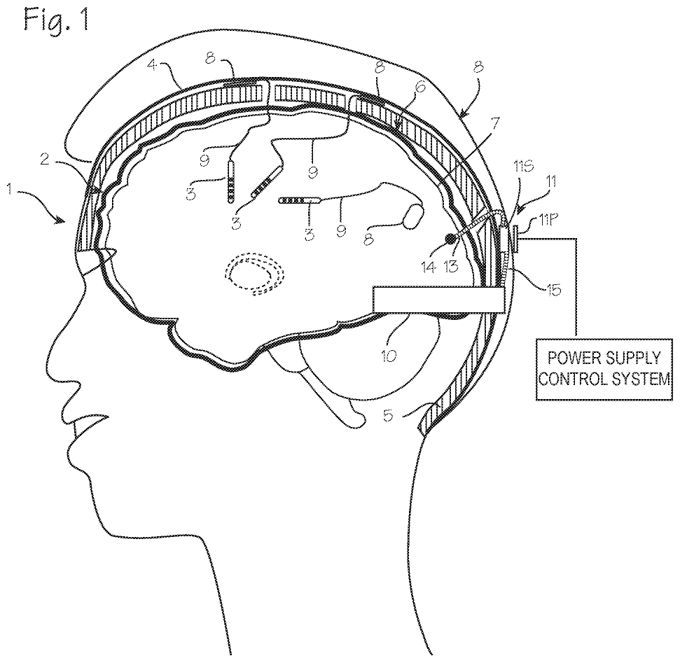

FIG. 1 illustrates a system for treatment of movement disorders using senor probes and stimulation probes operated by an external control system. A patient 1 with a condition requiring deep brain stimulation (DBS) or diagnosis of a condition of the brain 2 is illustrated. FIG. 1 shows the placement of a plurality of probes 3. The probes may be stimulation probes, without sensing capability, or sensor probes without stimulation capability, or combined sensor and stimulation probes. The sensor probes and stimulation probes can be inserted entirely within the brain at various positions. FIG. 1 also illustrates the scalp 4, the skull 5 which is beneath the scalp, the dura 6 which is beneath the skull and the cerebral cortex 7 which is beneath the dura.

The probes 3 of FIG. 1 are attached to electrodes 8, which are implanted preferably in a subcutaneous location (under the skin, superficial to the skull) under the scalp, through conductors 9. The probes have been inserted into the brain of the patient through openings (typically, burr-holes) in the skull, and driven through the brain and deposited at a location determined by a surgeon and known to effect target disorders, or known to produce signals indicative of target disorders. The electrodes 8 have been implanted subcutaneously, but may be installed supra-cutaneously or within the burr-holes.

To establish a power circuit from a power source to the probes, and/or communicate sensor data from the probes to a control system, in conjunction with the electrodes 8, the patch electrode 10 is disposed on the scalp, supracutaneously on the scalp, or subcutaneously under the scalp. The patch electrode is, preferably, located such that it is not in direct physical contact with the electrodes 8, and is spaced from the electrodes 8. The patch electrode 10 is connected to the secondary (remote) coupling component 11S of a coupling assembly 11, and the primary (base) coupling component 11P is connected to the power supply and control system 12. Preferably, the coupling assembly is an inductive coupling assembly, comprising a pair of coils. The secondary (remote) coupling component 11S may, like the patch electrode, also be installed on the scalp, supra-cutaneously, or subcutaneously under the scalp. The primary (base) coupling component 11P may be placed in proximity to the secondary coupling using a magnetic attachment or other releasable attachment means (secured to a headband, glued to the overlying skin) or non-releasable means (stitched to the scalp, nailed or screwed to the skull, or other means not considered “releasable attachment means and require tools for removal). An insulated conductor 13 extends from the secondary (remote) coupling component 11S, through a burr-hole and into the brain (including the dura and the cortex), and may be an insulated wire and include an electrode 14 at its distal end (a conductive wire, insulated or bare, will suffice). An additional conductor 15 connects the secondary (remote) coupling component 11S to the patch electrode 10. The control system is configured to provide power to the probes, for stimulation of brain tissue proximate the probes, and receive sensor data from the probe. The control system may operate as a DBS pulse generator, with or without further functionality. The control system 12 may be further programmed to analyze the sensor data and modify control signals to the probes to control the stimulation provided by the probes in response to the sensor data. The control system or console used to control the system and/or receive and store EEG data from the electrodes and provide bi-directional communications through the coupling, and provide stimulation pulses to the probed, may be implemented on a dedicated console such as the Kohden Neurofax EEG-1200 console (without all the wires), and EEG monitoring system, a DBS pulse generator such as a Vercise Genus™ implantable pulse generator, a general-purpose computer, or a mobile phone or tablet.

The probe electrodes and electronics may be operable to detect native biological brain signals (electrical activity of the brain), and generate electronic signals corresponding to native biological brain signals and transmit those electronic signals to the control system through the electrode 8 and patch electrode 10 and power coupling 11. The control system 12 is operable to receive electronic signals corresponding to native biological brain signals from the probes and, optionally, to interpret those signals. The control system may also store this data, or transmit it elsewhere for storage and review. For example, the control system may be configured, with appropriate programming, to analyze the electronic signals corresponding to native biological brain signals and determine whether the native biological brain signals are within a predetermined band of native biological brain signals, or above or below a predetermined threshold for the native biological brain signals, or characteristic of movement disorders. The control system is may also be operable to generate and transmit control signals to the probes, to cause the probes to transmit stimulation pulses to structures within the brain to effect therapeutic changes in native biological brain signals. The native biological brain signals of interest correspond to motor deficiencies, which may include Parkinson's Disease, epilepsy, essential tremors and/or dystonia. The native biological brain signals may also be signals such as abnormal hyperactivity in Broadmann's area that correspond to mood disorders such as depression. Stimulation can also be applied to areas of the dorsolateral prefrontal and lateral orbitofrontal cortex for associative diseases involved in cognition or memory, to the limbic and paralimbic cortex, hippocampus and amygdala for the treatment of limbic ailments such as OCD or for treatment of pain management, stroke rehabilitation and cognition impairment. Monitoring and stimulation can be in different locations of the brain, using some of the probes 3 in a sensing mode and using some of the probes 3 in a stimulation mode, and/or using some of the probes in both modes.

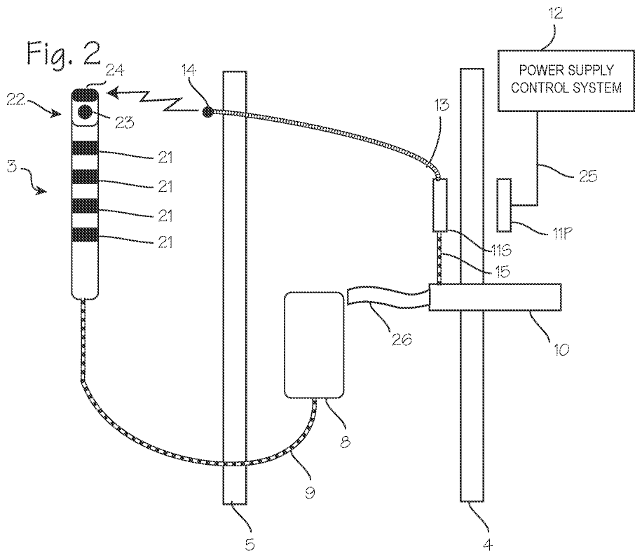

FIG. 2 illustrates the probe 3 and subcutaneous electrode 8. The probe 3 includes several sensor/stimulation electrodes 21, and may also include an LED assembly 22 which includes an LED 23, and a power/signal contact 24 for providing power to the electrodes 21 and the LED 23 or transmitting electronic signals to the control system/console. The sensor/stimulation electrodes are connected to the subcutaneous electrode 8, and may be operated, in conjunction with the subcutaneous electrode 8 to apply electrical stimulation to brain tissue or sense electrical signals of the brain, or both. The LED may be used to provide photonic stimulation of the brain. FIG. 2 also shows the conductor 9 and subcutaneous electrode 8. The subcutaneous electrode 8, which is preferably configured for implantation under the scalp, may consist of a bare conductive plate (metal such as stainless steel, or carbon, carbon fiber, glassy/pyrolytic carbon or graphene), but may also consist of a conductive plate covered in thin layer of insulation. The subcutaneous electrode 8 may also comprise a near field communication coil (an NFC tag) configured to communicate with the control system through a corresponding emitter and NFC controller of the control system (which may be housed proximate the patch electrode or housed in a separate appliance). The conductor 9 also functions as a tether for retrieval of the probe from the brain. The power/signal contact 24 on the probe tip may be bare, uncoated metal or other conductive material, or may be coated with a non-insulating coating with biologic effect, or covered in an insulating layer and work in conjunction with an insulated subcutaneous electrode 8 for capacitive coupling of both components of the probe to the conductor 13 of the secondary coil 11S.

The circuit established by the patch electrode 10, sensor/stimulation 21 electrodes and subcutaneous electrodes 8 is completed by the body tissue disposed between the various components. FIG. 2 illustrates, in addition to the probe 3, the components of the circuit and the circuit path for power and sensor signals. As shown in FIG. 2, power is supplied by the power supply 12, and is transmitted through conductor 25 to the primary (base) coupling component 11P, over the gap to the secondary (remote) coupling component 11S, and through the conductor 13 passing through the burr-hole and extending toward the brain 2 (proximate the dura 6, and preferably extending into the 7 cortex). The conductor 13 terminates proximate the dura, so the power and sensor signals pass through the cortex to the tip electrode 24 on the tip of the probe 3, to power electronics on the probe associated with the LED or the stimulation electrodes. The circuit is completed through the conductor 9 to the subcutaneous electrode 8, through subcutaneous tissue 26 of the scalp (which may include connective tissue, epicranial apaneurosis, areolar connective tissue, and periosteum), to the patch electrode 10, and through conductor 15 to the secondary (remote) coupling component 11S, across the air gap to the primary (base) coupling component 11P and thence to the power supply and control system. Preferably, there is no wired connection between the intracranial electrode 14 and the probe, and there is no wired connection between the subcutaneous electrode and the patch electrode. The skull serves as an insulator, separating the electrical conductor 13 terminus inside the skull (within the brain) from the subcutaneous electrode subcutaneous electrode 8 and patch electrode 10 which are both located superficial to the skull.

In use, the components of the system are installed in and on the patient, as described above. The control system may be operated to cause the probes to apply stimulation to structures in the brain proximate the probes. The stimulation may be a voltage applied through one or more of the electrodes 21. This voltage may be applied in bipolar mode (with a voltage differential between the electrodes) or monopolar mode (with a voltage differential applied between the patch electrode and the electrodes 21). The stimulation may be light emitted by the LED 23. The control system may also be operated to cause the probes to sense electrical signal of the brain, particularly electrical signals associated with motor or mood disorders, and transmit those signals to the control system. The control system may be operated to generate and display images corresponding to the sensed electrical signal, forward those signals to other computers for storage and analysis, or analyze those signals and determine, based on those signals, whether to apply stimulation through the probe to affect the brain.

The probes are implanted within the brain and positioned in multiple regions of the brain subject to stimulation to affect symptoms of a disease such as Parkinson's disease, epilepsy, essential tremor and dystonia. The sensor components of the probes are operated to generate electronic signals corresponding to the sensed biological signals of the patient's brain. The electronic signals are transmitted to the control system through the circuit illustrated in the Figures. To provide stimulation to the brain through stimulation components of the probes, the control system may be programmed and configured to interpret whether the measured signal is within a predetermined band of signal readings, or above or below a predetermined threshold for the signal readings. The control system may then also be operable to generate and transmit control signals to the probes, to cause the probes to transmit stimulation pulses to structures within the brain if the signal readings are determined to be outside of a predetermined range to effect therapeutic changes in native biological brain signals. The control system may thus be programmed and operable to cause the probes to deliver a prescribed dosage of stimulation impulses to treat a variety of conditions and diseases such as Parkinson's disease, epilepsy, essential tremor and dystonia.

Sensor probes 3 inserted on or within the brain 2 detect and/or record signals linked to symptoms exhibited within the brain. The sensor probes detect EEG, ECoG, AP, LFP or other detectable bio-signals. The sensor probes transmit the electronic signal corresponding to the sensed bio-signal to the receiver of the control system. The control system processes the electronic signal and is operable to transmit control signals to the stimulation probes to cause the stimulation probes to apply stimulation in response to variations in the sensed signals. Depending on the placement, the sensor probes are operable to sense signals from the thalamus, STN, cortex or other associated structures of the brain, which are indicative of the conditions treated (signals indicative of reduced or increased unwanted motor activity in the patient, for example). Adjustment in the stimulation provided by the stimulation probes can also be made through operator input to the control system, in response to sensed signals from the sensor probes, for example in response to data provided by the control system through an output such as a display screen or audio speakers. Adjustment in the stimulation provided by the stimulation probes may also be made by the control system, without immediate operator input, if the control system is programmed to determine stimulation levels or patterns appropriate to apply or adjust in response to sensed signals from the sense probes. This real-time optimization would allow neurons the chance to rest and thus reduce overall deterioration over time.

For the purposes of the claims, we coin the term peri-cutaneous, to refer to supra-cutaneous and subcutaneous place (that is, “near” the skin), and clarify that subcutaneous refers to its normally understood meaning of placement under the skin, but superficial to the skull, and supra-cutaneous refers to its normally understood meaning of placement on the exterior surface of the skin (superficial to the skin) but not excluding the possibility of an intervening impedance matching substance, adhesive, cream or ointment or hair.

While the preferred embodiments of the devices and methods have been described in reference to the environment in which they were developed, they are merely illustrative of the principles of the inventions. The elements of the various embodiments may be incorporated into each of the other species to obtain the benefits of those elements in combination with such other species, and the various beneficial features may be employed in embodiments alone or in combination with each other. Other embodiments and configurations may be devised without departing from the spirit of the inventions and the scope of the appended claims.

I claim:

1. A system for applying stimulation to a brain of a patient, said system comprising:
a probe (3) configured for implantation in the brain of the patient, said probe comprising a means for stimulating brain tissue (21, 22), and a power/signal contact 24 for receiving power;
a subcutaneous electrode (8);
a first electrical conductor (9) connecting the probe (3) to the subcutaneous electrode (8);
an inductive coupling assembly (11) comprising a primary (base) coupling component (11P), secondary (remote) coupling component (11S), said secondary (remote) coupling component (11S) configured for subcutaneous placement between the scalp and skull of the patient, said primary (base) coupling component (11P) configured for coupling with the secondary (remote) coupling component (11S) while disposed supra-cutaneously and proximate the secondary (remote) coupling component (11S);
a patch electrode (10) configured for subcutaneous placement between the scalp and skull of the patient;
a second electrical conductor (13), said second electrical conductor (13) configured for electrically connecting the secondary (remote) coupling component (11S) to the brain of the patient;
a third electrical conductor (15), said third electrical conductor (15) connecting the secondary (remote) coupling component (11S) to the patch electrode; and
a power supply (12) operable connected to the primary (base) coupling component (11P).

2. The system of claim 1, wherein an electrical connection between the second electrical connector and power/signal contact (24) for receiving power does not include a wired connection.

3. The system of claim 1, wherein an electrical connection between the subcutaneous electrode (8) and the patch electrode (10) does not include a wired connection.

4. The system of claim 1 wherein the means for stimulating brain tissue comprises a plurality of stimulation electrodes (21) disposed on the probe (3) and operable to apply electrical stimulation to the brain tissue proximate the probe (3).

5. The system of claim 1 wherein the means for stimulating brain tissue comprises a light source (23) operable to apply light to brain tissue proxime the probe (3).

6. The system of claim 1 wherein the primary (base) coupling component (11P) comprises a primary coil of an inductive coupling (11) and the secondary (remote) coupling component (11S) comprises a secondary coil of an inductive coupling (11).

7. The system of claim 1 wherein the first electrical conductor (9) is configured as a tether for retrieval of the probe (3) from the brain.

8. The system of claim 1 wherein the primary (base) coupling component (11P) is releasably attachable to the secondary (remote) coupling component (11S).

9. The system of claim 1 wherein the primary (base) coupling component (11P) is disposed on a headband, whereby the primary (base) coupling component (11P) may be secured to the patient proximate the secondary (remote) coupling component (11S).

10. The system of claim 1, wherein there is no wired connection between the second electrical conductor (13) and the probe (3), and wherein there is no wired connection between the subcutaneous electrode (8) and the patch electrode.

11. The system of claim 1, further comprising:
a control system 12 operable to control the power supply (12) to provide power to the probe (3) through the inductive coupling assembly and control the probe (3) to cause the probe (3) to detect electrical activity of brain proximate the probe (3) and generate electronic signals corresponding to electrical activity and transmit those signals to a control system (12).

12. The system of claim 11, wherein:
the power supply (12) provides power through a circuit established from the secondary (remote) coupling component (11S), through the second electrical connector, through brain tissue to the power/signal contact, through the first electrical conductor (9) to the subcutaneous electrode (8), through scalp tissue to the patch electrode, and through the third electrical conductor (15) to the secondary (remote) coupling component (11S).

13. A system for sensing electrical activity of a brain of a patient, said system comprising:
a probe (3) configured for implantation in the brain of the patient, said probe (3) comprising means for sensing electrical activity of brain tissue (21, 22), and a signal contact 24 for transmitting signals corresponding to the electrical activity to a control system;
a subcutaneous electrode (8);
a first electrical conductor (9) connecting the probe (3) to the subcutaneous electrode (8);
an inductive coupling assembly (11) comprising a primary (base) coupling component (11P), secondary (remote) coupling component (11S), said secondary (remote) coupling component (11S) configured for subcutaneous placement between the scalp and skull of the patient, said primary (base) coupling component (11P) configured for coupling with the secondary (remote) coupling component (11S) while disposed supra-cutaneously and proximate the secondary (remote) coupling component (11S);
a patch electrode (10) configured for subcutaneous placement between the scalp and skull of the patient;
a second electrical conductor (13), said second electrical conductor (13) configured for electrically connecting the secondary (remote) coupling component (11S) to the brain of the patient;
a third electrical conductor (15), said third electrical conductor (15) connecting the secondary (remote) coupling component (11S) to the patch electrode (10);
a control system operable connected to the primary (base) coupling component (11P), said control system operable to control the probe (3) to obtain signals corresponding to electrical activity of the brain and transmit electronic signals corresponding to the signals corresponding to electrical activity of the brain to the control system through the inductive coupling.

14. The system of claim 1, wherein an electrical connection between the second electrical connector and signal contact for transmitting signals does not include a wired connection.

15. The system of claim 1, wherein electrical connection between the subcutaneous electrode (8) and the patch electrode (10) does not include a wired connection.

16. The system of claim 1 wherein the means for sensing electrical activity of brain tissue comprises a plurality of electrodes (21) disposed on the probe (3) and operable to sense electrical activity of brain tissue proximate the probe (3).

17. The system of claim 1 wherein the primary (base) coupling component (11P) comprises a primary coil of an inductive coupling (11) and the secondary (remote) coupling component (11S) comprises a secondary coil of an inductive coupling (11).

18. The system of claim 1 wherein the first electrical conductor (9) is configured as a tether for retrieval of the probe (3) from the brain.

19. The system of claim 1 wherein the primary (base) coupling component (11P) is releasably attachable to the secondary (remote) coupling component (11S).

20. The system of claim 1 wherein the primary (base) coupling component (11P) is disposed on a headband, whereby the primary (base) coupling component (11P) may be secured to the patient proximate the secondary (remote) coupling component (11S).

21. The system of claim 1, wherein there is no wired connection between the second electrical conductor (13) and the probe (3), and wherein there is no wired connection between the subcutaneous electrode (8) and the patch electrode (10).

22. The system of claim 1, further comprising:
a control system 12 operable to control the probe (3) through the inductive coupling assembly to cause the probe (3) to detect electrical activity of the brain proximate the probe (3) and generate electronic signals corresponding to the electrical activity of the brain and transmit those signals to a control system.

23. The system of claim 11, wherein:
the control system 12 obtains the electronic signals corresponding to the electrical activity of the brain through a circuit established from the secondary (remote) coupling component 11S, through the second electrical connector, through brain tissue to the power/signal contact, through the first electrical conductor (9) to the subcutaneous electrode (8), through scalp tissue to the patch electrode (10), and through the third electrical conductor (15) to the secondary (remote) coupling component (11S).

24. A method for applying stimulation to a brain of a patient, said method comprising the steps of:
implanting a probe (3) configured for implantation in the brain of the patient, said probe (3) comprising a means for stimulating brain tissue, a power/signal contact for receiving power;
said probe (3) being connected to a subcutaneous electrode (8) through a first electrical conductor (9);
implanting the subcutaneous electrode (8) under the scalp of the patient, with the first electrical conductor (9) passing through the skull;
affixing a secondary (remote) coupling component (11S) of an inductive coupling assembly comprising a secondary (remote) coupling component (11S) and primary (base) coupling component (11P) to the scalp of the patient, peri-cutanously, and placing the primary (base) coupling component (11P)proximate the secondary (remote) coupling component (11S);
implanting a patch electrode (10) subcutaneously between the scalp and skull of the patient;
electrically connecting the secondary (remote) coupling component (11S) to the brain of the patient through a second electrical connector (13);
electrically connecting the secondary (remote) coupling component (11S) to the patch electrode (10) through a third electrical conductor (15);
connecting a power supply (12) to the primary (base) coupling component (11P); and
operating the power supply (12) to cause the probe (3) to apply stimulation to brain tissue proximate the probe (3), through a circuit established from the secondary (remote) coupling component (11S), through the second electrical connector, through brain tissue to the power/signal contact, through the first electrical conductor (9) to the subcutaneous electrode (8), through scalp tissue to the patch electrode (10), and through the third electrical conductor (15) to the secondary (remote) coupling component (11S).

25. The method of claim 12 further comprising wherein:
the step of causing the probe (3) to apply stimulation to the brain comprises causing the probe (3) to apply electrical stimulation to the brain through a stimulation electrode disposed on the probe (3).

26. The method of claim 14 further comprising wherein:
the step of causing the probe (3) to apply stimulation to the brain comprises energizing an LED on the probe (3).

27. A method of treating Parkinson's disease, epilepsy, essential tremor or dystonia, said method comprising use of the method of claim 14.

28. A method for sensing electrical activity a brain of a patient, said method comprising the steps of:
implanting a probe (3) configured for implantation in the brain of the patient, said probe (3) comprising a means for sensing electrical activity of brain tissue, and a power/signal contact for receiving power;
said probe (3) being connected to a subcutaneous electrode (8) through a first electrical conductor (9);
implanting the subcutaneous electrode (8) under the scalp of the patient, with the first electrical conductor (9) passing through the skull;
affixing a secondary (remote) coupling component 11S of an inductive coupling assembly comprising a secondary (remote) coupling component (11S) and primary (base) coupling component (11P) to the scalp of the patient, peri-cutanously, and placing the primary (base) coupling component (11P) proximate the secondary (remote) coupling component (11S);
implanting a patch electrode (10) subcutaneously between the scalp and skull of the patient;
electrically connecting the secondary (remote) coupling component (11S) to the brain of the patient through a second electrical connector (13);
electrically connecting the secondary (remote) coupling component (11S) to the patch electrode (10) through a third electrical conductor (15);
connecting a power supply (12) to the primary (base) coupling component (11P); and
operating the power supply (12) to cause the probe (3) to detect electrical activity of brain proximate the probe (3) and generate electronic signals corresponding to electrical activity and transmit those signals to a control system, through a circuit established from the secondary (remote) coupling component (11S), through the second electrical connector (13), through brain tissue to the power/signal contact, through the first electrical conductor (9) to the subcutaneous electrode (8), through scalp tissue to the patch electrode (10), and through the third electrical conductor (15) to the secondary (remote) coupling component (11S).
